(19) United States
(12) Patent Application Publication
(10) Pub. No.: US 2016/0296728 A1
(43) Pub. Date: Oct. 13, 2016

(54) CATHETER HAVING SLOTTED CANNULA AND NEEDLE RETRACTION MECHANISM

(71) Applicant: Emmett L. Smith, Renton, WA (US)

(72) Inventor: Emmett L. Smith, Renton, WA (US)

(21) Appl. No.: 15/098,296

(22) Filed: Apr. 13, 2016

Related U.S. Application Data
(60) Provisional application No. 62/146,862, filed on Apr. 13, 2015.

(51) Int. Cl. A61M 25/06; A61M 39/10; A61M 39/24; A61M 1/36; A61M 25/00

(52) U.S. Cl. CPC A61M 25/0631; A61M 1/3659; A61M 25/0026; A61M 25/0693; A61M 39/24; A61M 25/0043; A61M 25/007; A61M 25/0637; A61M 39/10; A61M 2025/0039

(57) ABSTRACT

An innovative intravenous catheter is described. The inventive catheter comprises an introduction needle, a cannula portion having one or more slots, through which the introduction needle extends when deployed, an in-dwelling check valve, a needle retraction mechanism, and a safety enclosure to contain the introduction needle when retracted. The in-dwelling check valve may further allow a needle from syringe or intravenous drip to be inserted from the rear end of the check valve and extend into the flash chamber to introduce other fluids, and function as a second fluid line. The one or more slots disposed along the cannula wall and may extend at least a portion of the length of the cannula portion, and function as to increase blood flow into the catheter, reducing the pressure required to flow blood into the catheter. The introduction needle may be caused to retract into a safety enclosure affixed to the inventive catheter.

### CROSS-REFERENCE TO PRIORITY APPLICATIONS

This non-provisional patent application claims the benefit of U.S. Provisional Application No. 62/146,862 filed on Apr. 13, 2015.

### FIELD OF THE INVENTION

This innovation relates to intravenous catheters and related catheter types, in particular for use in kidney dialysis and other forms of dialysis.

### BACKGROUND

Modern needles used for kidney dialysis and similar applications have not changed in basic design for decades. Some problems involving current needles is the chance of “backwall”, or infiltration involving going through the blood vessel completely. During a dialysis session, a patient may be completely forbidden from moving and even speaking or coughing, for fear that the needle becoming suctioned to the vessel wall. Dialysis needles can furthermore be difficult to insert and may nick the vessel upon insertion. Thirdly, current needles have no means to easily prevent back-flow of blood to the rear of the catheter.

### SUMMARY

The innovation described herein is an inventive intravenous dialysis catheter that addresses the concerns raised above. The inventive dialysis catheter comprises an introduction needle, a cannula portion, having a plurality of slots or elongated apertures, through which the introduction needle extends when deployed, an in-dwelling check valve, a needle retraction mechanism, and a safety enclosure to contain the introduction needle when retracted. In some embodiments of the innovative catheter, one or more elongated apertures or slots are disposed along the cannula wall and may extend substantially the length of the cannula portion. In other embodiments, one or more elongated apertures or slots disposed along the cannula wall and may extend a partial length of the cannula, for example, from one-quarter to one-half the length of the cannula portion of the innovative catheter. The novel addition of elongated apertures along the cannula wall function to increase blood flow into the catheter and to reduce the pressure required to flow blood into the catheter compared to the traditional single-orifice cannula in ubiquitous use. Once the needle retraction mechanism is engaged, the introduction needle is caused to retract into a safety enclosure affixed to the inventive catheter. Short apertures may aid in cannula retention in the patient's blood vessel once inserted therein. The use of “slots” and “elongated apertures” are used interchangeably throughout this disclosure.

Further embodiments of the innovative catheter include different forms of needle retraction actuation. One embodiment uses a spring-loaded release mechanism, the needle may be affixed to a tension spring or a compression spring. In one described embodiment, a tension spring is used so that when the needle is deployed, the spring is stretched. A slider attachment is also affixed to the needle to be lodged in a notch or other means of retention disposed near the top of the safety enclosure, which is preferably a vial-shaped container having a mouth at one end. Slots may be formed in the walls of the safety enclosure to allow the slider to slide toward the bottom of the safety enclosure when released from the notch, or other retention means by manual manipulation. Tension in the spring pulls the needle into the safety enclosure and tends to retain it therein.

In a further embodiment, no spring is used, but a finger-actuated slider mechanism functions by having a finger grip affixed to a support block affixed to the needle. The support block comprises a toggle cross piece that is affixed to the support block, but is disposed on the exterior of the safety container wall. The support block therefore extends through the wall via a slot that is formed in the wall of the safety enclosure. In this embodiment, the needle is deployed for insertion into a patient by extending forward through the cannula, and is retracted into the safety container by finger actuation. Once the needle is retracted, the safety enclosure may be detached from the inventive catheter for safe needle disposal.

The in-dwelling check valve comprises a two or more flexible flaps similar to those of a mitral valve. The novel check valve is adapted to allow the introduction needle to extend through the flaps when deployed. When the needle is retracted, the flaps close to seal the front end of the in-dwelling check valve. The innovation allows introduction of other fluids into the catheter flash chamber by attachment of a drip line or by insertion of a needle from a syringe, where the needle may be inserted into the check valve and extend into the flash chamber.

### BRIEF DESCRIPTION OF THE DRAWINGS

FIG. 1a. Overview of inventive catheter assembly, showing introduction needle extended for deployment. Inset shows slotted cannula detail.

FIG. 1b. Overview of the inventive catheter assembly, showing similar detail to that in FIG. 1a, but now showing short slots along the cannula wall.

FIG. 1c. Overview of inventive catheter assembly, showing slotted cannula detail introduction needle retracted. Slots run the substantially the length of the cannula.

FIG. 1d. Overview of inventive catheter assembly, showing slotted cannula detail introduction needle retracted. Slots here are shortened, running a fraction of the length of the cannula.

FIG. 2a. One embodiment of safety enclosure having slots for one embodiment of needle retraction mechanism

FIG. 2b. View of safety enclosure embodiment shown in FIG. 2a. Needle retraction mechanism is shown with needle retracted into the safety enclosure.

FIG. 2c. View of safety enclosure embodiment shown in FIG. 2a. Needle retraction mechanism is shown with needle extending from the safety enclosure.

FIG. 3a. Alternative embodiment of safety enclosure and needle retraction mechanism, needle extended. Side view.

FIG. 3b. Alternative embodiment of safety enclosure and needle retraction mechanism, needle extended. Top view.

FIG. 3c. Alternative embodiment of safety enclosure and needle retraction mechanism, needle retracted. Side view.

FIG. 4. Cross-sectional view of support block assembly.

FIG. 5a. Oblique view of in-line check valve details.

FIG. 5b. Oblique view of in-line check valve deployed in a section of the flash chamber of the inventive catheter with needle extending through the flexible flaps of the in-line check valve.

FIG. 5c. Oblique view of in-line check valve deployed in a section of the flash chamber of the inventive catheter with needle withdrawn.

### DETAILED DESCRIPTION

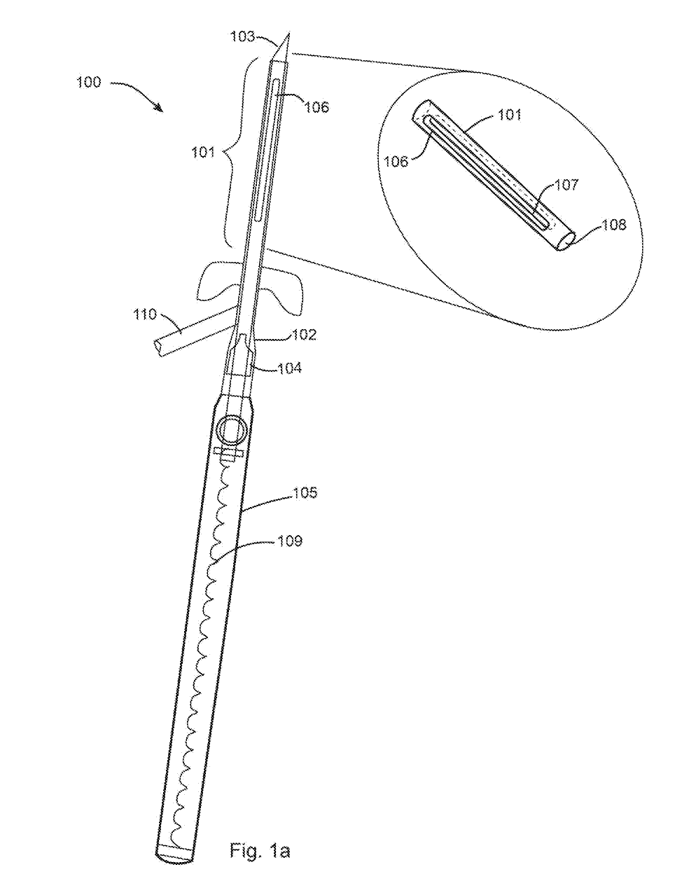

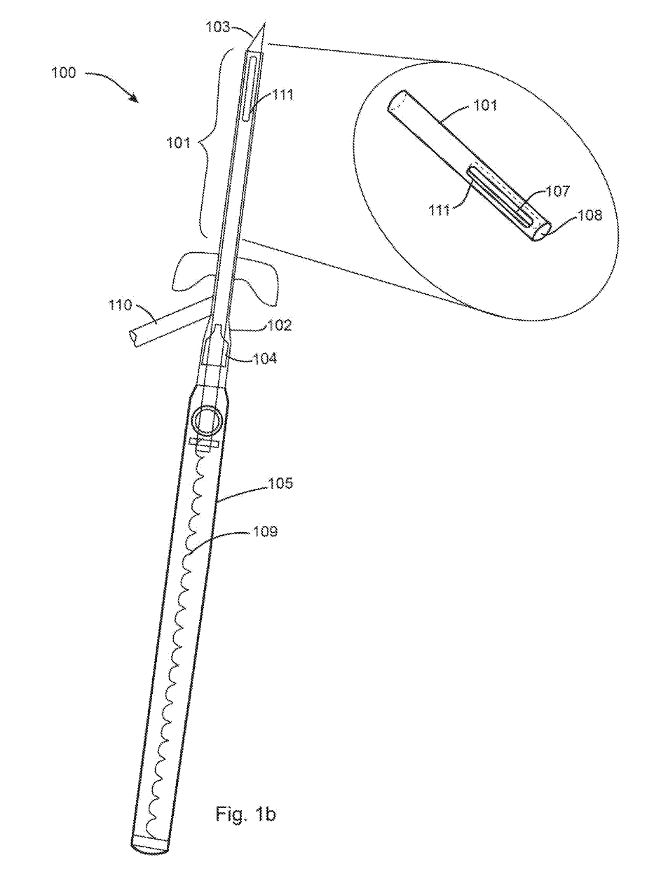

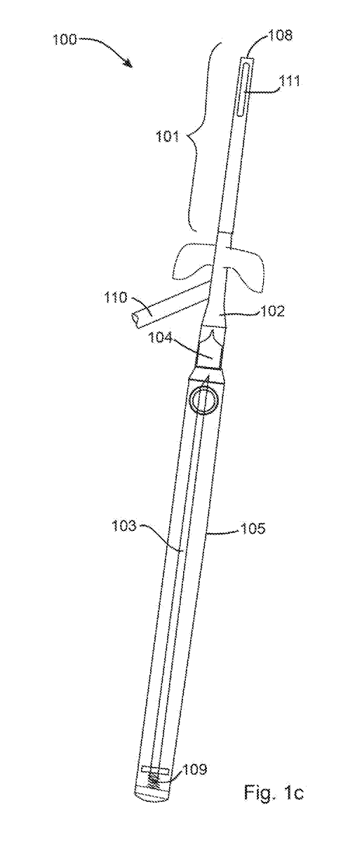

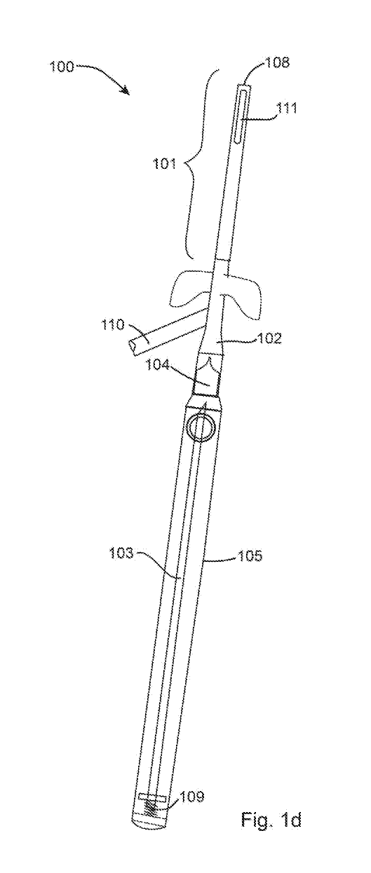

Referring to FIG. 1a, an assembled view of the inventive catheter 100 is shown. The inventive catheter comprises a slotted cannula portion 101, flash chamber 102, introducer needle 103, extending through check valve 104 and into safety needle enclosure, or containment vial, 105. The inventive catheter assembly further comprises a novel check valve 104 that seats within flash chamber 102. One object of the instant innovation is to provide increased blood flow into and out of the catheter at lower pumping pressures currently needed to maintain a given flow rate in intravenous therapies such as blood dialysis. To this end, the inventive catheter assembly features at least one elongated aperture running substantially along the length of the wall of cannula 101.

The inset of FIG. 1a shows two elongated apertures or slots 106 and 107 disposed on opposite sides along the wall of the distal portion of cannula 101, in addition to the exit orifice 108 at the distal end of cannula 101. The one or more elongated apertures (slots) provide the advantage of increased blood or fluid flow from the blood vessel to the catheter, or from the catheter to the blood vessel, when the catheter is deployed intravenously. In other embodiments, more than two elongated apertures may be present. Embodiments possessing two elongated apertures are depicted in the drawings for clarity. Tubing connector 110 is also shown as a standard feature of most dialysis catheters for connection of drip line tubing or dialysis tubing.

Another embodiment of the innovative catheter is shown in FIG. 1b, where the elongated apertures or slots 111 are shortened relative to slots 106 in FIG. 1. Apertures 111 may have a length ranging from one-quarter to one-half the length of the cannula, and disposed in the distal portion of the cannula. The shorter apertures or slots may provide for less chance of dislodgement of the cannula from a patient's blood vessel during a dialysis treatment.

Currently, needles used IV catheters rely solely on the single port disposed at the distal end of the cannula for fluid exchange in conventional catheters. The single port is moreover of small diameter, necessitating high pressure to force fluids out of the port (108) at an adequate rate, as the small port opening can act as a bottleneck in the fluid path, as in standard kidney dialysis treatment. Blood being forced through the narrow port at relatively high pressures can increase shear forces on the red and white blood cells due to high exit velocities, frequently leading to rupture of the red corpuscles. As a result, blood clots may form at or near the catheter opening. The instant innovation alleviates the pressures required to pump blood into and out of a dialysis machine at a given flow rate. The innovation permits higher flow volumes to be pumped at lower pressures by virtue of the increased cross sectional area fluidic openings, all the while maintaining cell integrity.

Another object of the instant innovation is to provide a built-in safety feature for post-sharp handling. To this end, introducer needle 103 may be affixed to tension spring 109 at one end to needle 103 near its proximal end in some embodiments. Tension spring 109 is shown in the extended position in FIGS. 1a and 1b, allowing the tip of introducer needle 103 to protrude from the exit port 108 at the distal end of inventive cannula 101. Equally, a compression spring may be used whereby the compression spring may be affixed to the needle near the proximal end. The proximal end of the needle is defined as the blunt end of the needle, or the end opposite the sharp end (distal end). The second end of spring 109 is affixed to safety needle enclosure 105, which may be a detachable vial.

Some embodiments of the instant innovation comprise a release mechanism to cause spring 109 to retract introducer needle 103 into needle safety enclosure 105, as is shown in FIG. 1c. The innovative arrangement permits introducer needle 103 to be safely withdrawn and stowed after positioning the inventive intravenous catheter in the blood vessel, without any chance for direct contact with the sharp by the medical practitioner, avoiding accidental sticking and contamination. Moreover, introducer needle 103 is not left in the catheter once it is inserted into the blood vessel, allowing the patient to move the limb or other body part more freely without worry of inadvertent puncture of the vessel wall by a still protruding needle that is left protruding from the exit port. The needle safety enclosure 105 may be detached from the inventive catheter 100 and capped to safely contain the needle, and prevent accidental sticking of the medical practitioner or other personnel. Fluid line attachment 110 is disposed along the wall of flash chamber 102 between the in-dwelling check valve 104 and cannula 102.

In FIG. 1d, the embodiment comprising shortened elongated apertures 111 is depicted with introducer needle 103 retracted.

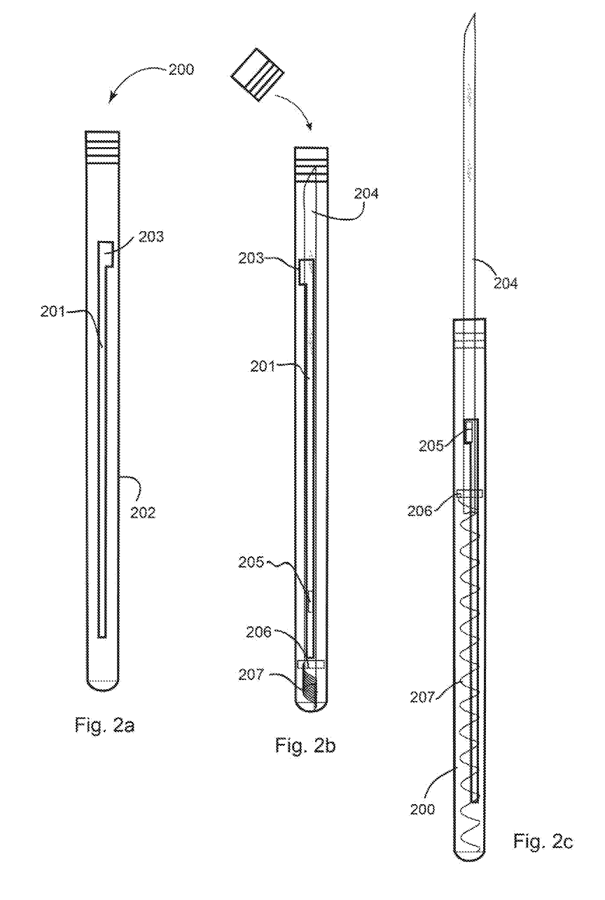

The release mechanism may be in the form of a button, as shown in FIGS. 1a and 1b, or may take on the other forms and styles. In one embodiment, shown in FIG. 2a, safety container 200 comprises two slots 201 (only one shown) that are disposed substantially axially along opposite sides of wall 202 of needle safety enclosure 200, which in this example is one that assumes a substantially vial-like tubular shape. In the embodiment shown, a notch 203 is disposed at one end of each of slots 201, where an end portion or segment of slots 201 is widened. Other means of retention or other detent mechanisms may be used. Slots 201 in combination with notches 203 may function as a simple slide track and release mechanism for tension (or compression) spring 105 shown in FIGS. 1a and 1b. Notch 203 may take the form of another means of retention for a slider attachment that may be affixed to the needle.

FIG. 2b shows an example of the function of slots 201 as a means to retract needle 204 via a tension spring release mechanism. In this depiction, introduction needle 204 is shown extending out of the mouth of container 200. Affixed to needle 204 is slider 205, which is shown to have wings that extend laterally from the body of slider 205. In this example, slider 205 is shown to be disposed near the proximal end of needle 204. Slider 205 may have wings that extend through slots 201, which may be manipulated by grasping with fingers and pushing or pulling slider 205 along slots 201. In addition, fixture 206 may be affixed to needle 204, preferably near the proximal end, to provide an attachment structure for tension spring 207, shown anchored at one end at the bottom of safety enclosure 200, and extending along the length of enclosure 200 to terminate at fixture 206 in a cocked position.

In some embodiments, enclosure 200 is a detachable vial or similar shape, comprising a mouth end that may be removably affixed to the rear of the flash chamber of the inventive catheter. Slider 205 may be caused to slide along slot 201 toward the mouth of container 200 to pull needle 204 into safety enclosure 200. Since slider 205 is affixed to needle 204, the action of sliding wing 205 along the slots also may be used to push needle 204 in the direction of the mouth of container 200, allowing needle 204 to protrude from container 200. This is depicted in FIG. 2c. When wing 205 reaches the end of slot 201, it may be seated in notch 203, where notch 203 restrains the tendency of spring 207 to pull back on needle 204, causing it to be retracted into container 200. Other more complex detention mechanisms may be substituted in other embodiments without departing from the scope and spirit of the innovation. This latter position is shown in FIG. 2c, where needle 204 has been released by moving wing 205 out of notch 203 and repositioning it back in slot 201. Tension from extended spring 207 takes over and retracts needle 205 back into container 200, and spring 205 is recoiled in its compact configuration at the bottom of container 200.

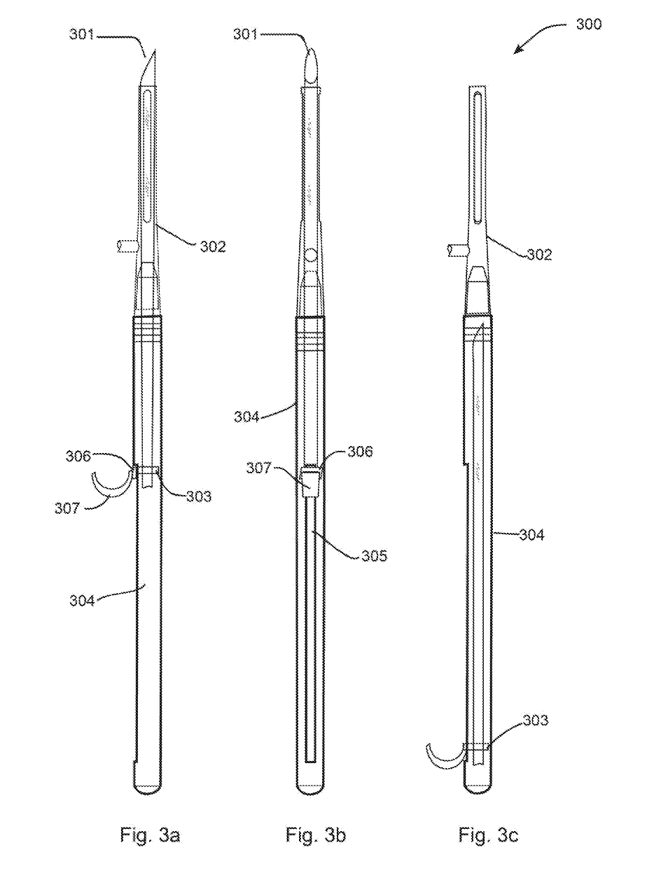

In another embodiment, a finger-actuated sliding needle support and needle combination, as shown in FIG. 3a. In this embodiment, shown in side view, introducer needle 301 is also retractable, but the retraction mechanism does not rely on spring actuation. When the catheter 300 is deployed, the needle is in the forward position, extending through the tip of the cannula 302 for piercing skin and blood vessel wall. Once the cannula 302 is inserted into the blood vessel lumen, the needle 301 may be retracted to allow the patient to move the limb without fear of the needle piercing or tearing the blood vessel wall while the catheter 300 is in place. The blunt end of needle 301 is endowed with a sliding support block 303 disposed thereupon, where needle 301 may be inserted and extend therethrough. Support block 303 may be affixed to needle 301 in a variety of other ways; it is to be understood that the particular embodiment shown in FIG. 3a is exemplary only.

Support block 303 is further adapted to be slideably seated within needle safety enclosure 304, having a stem protruding through slot 305 formed in the wall of needle safety enclosure 304. This is shown more clearly in FIG. 3b, where needle safety enclosure 304 is shown in top view to facilitate the description by rotating 90 degrees to expose slot 305 on top. Referring again to FIG. 3a, support block 303 is shown affixed to toggle crosspiece 306, which is disposed on the exterior of safety needle enclosure 304, and functions to slidely affix support block 303 against the interior wall of needle safety enclosure 304. Referring to FIG. 3b, toggle crosspiece 306 spans the width of slot 305 and may only be moved along the length of slot 305. Affixed to toggle crosspiece 306 is a finger placement 307, which serves to facilitate withdrawal of introducer needle 301 by using one hand to insert the catheter and then retract the needle while holding the catheter with the same hand. In FIG. 3c, introducer needle 301 is depicted in the retracted position, where it is now retracted into the interior of needle safety enclosure 304.

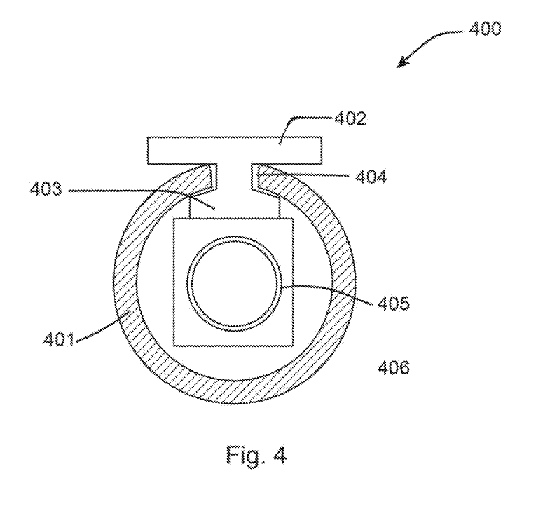

FIG. 4 shows a cross sectional detail of support block assembly 400, which is shown in cross section engaged with needle safety enclosure 401. Toggle crosspiece 402 is disposed on the exterior of wall of needle safety enclosure 401, where the support block 403 extends through slot 404 formed in the wall of needle safety enclosure 401. Support block 403 may comprise a lower portion through which introducer needle 405 extends, whereby support block 403 is affixed to introducer needle 405. Toggle crosspiece 402 may be integrally formed with support block 403. Alternatively, toggle crosspiece 402 may be affixed to support block 403, after insertion of introducer needle 405 to affix support block 403 to the introducer needle. The assembly may then be inserted into needle safety enclosure 401 and aligned with slot 404, where toggle crosspiece 402 may be fastened to the support block through slot 404 using a fastener such as a screw or bolt, or by application of an adhesive.

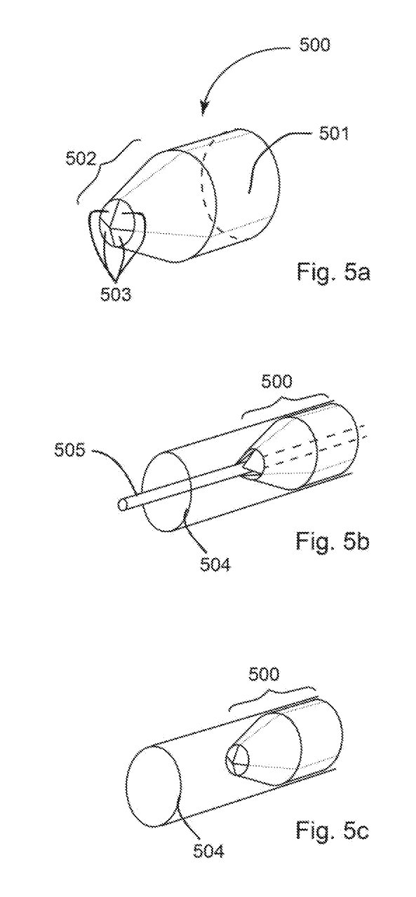

It is a further object of the instant innovation to provide an in-line check valve or one-way valve disposed within the catheter hub or flashback chamber, or in an appropriate preceding portion of the catheter construction. An exemplary embodiment of an in-line check valve is shown in FIG. 5a, where an example embodiment 500 is shown. Check valve 500 may comprise a substantially cylindrical open portion 501, terminated by a substantially conical or pyramidal nose section 502, which may further comprise a plurality of flexible flaps 503. The plurality of flexible flaps 503 may function as flaps in a mitral valve, allowing liquid flow primarily in one direction, which in the depiction of FIG. 5a, would flow from right to left, into the lumen of the catheter.

In some embodiments, in-dwelling check valve 500 is disposed between the cannula of the inventive catheter and the safety needle container. In FIG. 5b, a functional advantage of the design of the check valve is shown, where check valve 500 is seated in a segment of cannula flash chamber 504. Needle 505 is shown extending through the flaps 503, which are shown to separate to allow needle 505 to extend through. When the needle is withdrawn, flaps 503 close and form a seal to prevent fluids from flowing out the back end 501, as shown in FIG. 5c. In-dwelling check valve 500 may further allow a needle from syringe or IV drip to be inserted from the rear, or proximal, end of the check valve and extend into the flash chamber to introduce other fluids, and function as a second fluid line.

The particular embodiments described herein are but exemplary and are not to be construed as limiting the scope of the invention. Equivalent variations of the above embodiments not described herein are to be understood to be conceivable without departing from the scope and spirit of the innovation described herein, as claimed in the claims presented below.

1. A dialysis catheter, comprising:
(i) a cannula having a wall, a distal end and a proximal end, and one or more elongated apertures disposed along the wall of said cannula and extending at least a portion of the length thereof, said elongated apertures being distributed about the wall of said cannula portion, said cannula portion having coaxial lumen adapted to sheath an introducer needle, and said introducer needle having a proximal and distal end;
(ii) a flash chamber having a distal end and a proximal end, the distal end of said flash chamber integral with the proximal end of the cannula portion; and
(iii) a check valve disposed within the flash chamber.

2. The dialysis catheter of claim 1, wherein the elongated apertures disposed along the wall of the cannula extend substantially the entire length of the cannula from the distal end to the proximal end of said cannula.

3. The dialysis catheter of claim 1, wherein the elongated apertures disposed along the wall of the cannula extend between one quarter and one half of the length of the cannula and disposed along the distal portion of the wall of the cannula.

4. The dialysis catheter of claim 1, wherein said introducer needle is adapted to extend through the lumen of the cannula, and wherein the distal end of the introducer needle is adapted to protrude beyond the distal end of the cannula and to penetrate into a blood vessel of a patient.

5. The dialysis catheter of claim 1, further comprising a needle safety enclosure having a wall, a proximal end and a distal end, the distal end of said needle safety enclosure being removably affixed to the proximal end of the flash chamber,

6. The dialysis catheter of claim 5, wherein a spring having a first end is affixed along the proximal end of the introducer needle and having a second end affixed to the wall of the safety needle enclosure.

7. The dialysis catheter of claim 6, wherein the spring is a compression spring.

8. The dialysis catheter of claim 6, wherein the spring is a tension spring.

9. The dialysis catheter of claim 5, wherein at least one slot is disposed substantially axially along the wall of the safety needle enclosure.

10. The dialysis catheter of claim 9, wherein the at least one slot terminates in a notched end wherein the notch comprises a widened portion of the at least one slot.

11. The dialysis catheter of claim 10, wherein a slider having at least one wing extending laterally from the body of the slider is affixed to the introducer needle and is disposed within the needle safety enclosure, wherein the at least one wing of said slider extends through the at least one slot disposed axially along the wall of the safety needle enclosure and being slideably affixed thereto.

12. The dialysis catheter of claim 11, wherein the at least one wing of the slider is adapted to be pushed and pulled by digits of a human hand.

13. The dialysis catheter of claim 9, wherein a support block adapted to slideably seat within the needle safety enclosure is affixed to the introducer needle, and comprises at least one stem protruding through the at least one slot disposed axially along the wall of the safety needle enclosure, said at least one stem affixed to a toggle crosspiece disposed on the exterior surface of the wall of the needle safety enclosure and spanning the width of the at least one slot disposed therealong, such that the support block is slidably affixed to the wall of the needle safety enclosure.

14. The dialysis catheter of claim 12, wherein a finger placement is affixed to the toggle crosspiece.

15. The dialysis catheter of claim 1, wherein the check valve comprises a wall having a proximal end and a distal end, the distal end comprising two or more flaps extending distally toward the cannula, each flap comprising at least one complimentary edge, such that the complimentary edges are in contact when the two or more flaps are urged in the proximal direction, forming a seal and blocking fluids from moving in the proximal direction within the dialysis catheter, and parted when the two or more flaps are urged in the distal direction, permitting fluids to move in the distal direction within the dialysis catheter.

16. The dialysis catheter of claim 1, wherein the introducer needle extends through the check valve.

17. A dialysis catheter, comprising:
(i) a cannula having a wall, a distal end and a proximal end, and one or more elongated apertures disposed along the wall of said cannula and extending at least a portion of the length thereof, said elongated apertures being distributed about the wall of said cannula portion, said cannula portion having coaxial lumen adapted to sheath an introducer needle, and said introducer needle having a proximal and distal end;
(ii) a flash chamber having a distal end and a proximal end, the distal end of said flash chamber integral with the proximal end of the cannula portion; and
(iii) a needle safety enclosure having a wall, a proximal end and a distal end, the distal end of said needle safety enclosure being removably affixed to the proximal end of the flash chamber, wherein at least one slot is disposed substantially axially along the wall of the safety needle enclosure.
(iv) an in-dwelling check valve disposed within said flash chamber, the in-dwelling check valve having a proximal end and a distal end, and comprising two or more flaps disposed at the distal end, extending distally toward the cannula, each flap comprising at least one complimentary edge, such that the complimentary edges are in contact when the two or more flaps are urged in the proximal direction, forming a seal and blocking fluids from moving in the proximal direction within the dialysis catheter, and parted when the two or more flaps are urged in the distal direction, permitting fluids to move in the distal direction within the dialysis catheter.

18. The dialysis catheter of claim 17, wherein a spring having a first end is affixed along the proximal end of the introducer needle and having a second end affixed to the wall of the needle safety enclosure.

19. The dialysis catheter of claim 17, wherein a slider having at least one wing extending laterally from the body of the slider is affixed to the introducer needle and is disposed within the needle safety enclosure, wherein the at least one wing of said slider extends through the at least one slot disposed axially along the wall of the safety needle enclosure and being slideably affixed thereto.

20. The dialysis catheter of claim 17, wherein a support block adapted to slideably seat within the needle safety enclosure is affixed to the introducer needle, and comprises at least one stem protruding through the at least one slot disposed axially along the wall of the safety needle enclosure, said at least one stem affixed to a toggle crosspiece disposed on the exterior surface of the wall of the needle safety enclosure and spanning the width of the at least one slot disposed therealong, such that the support block is slidably affixed to the wall of the needle safety enclosure.
